(19) United States
(12) Patent Application Publication
(10) Pub. No.: US 2005/0025839 A1
(43) Pub. Date: Feb. 3, 2005

(54) Formulation approach to enhance transporter-mediated drug uptake

(76) Inventor: James Edward Polli, Ellicott City, MD (US)

Correspondence Address: James E. Polli, 3524 Bucks County Court, Ellicott City, MD, 21043, US

(21) Appl. No.: 10/898,771

(22) Filed: Jul. 26, 2004

Related U.S. Application Data
(60) Provisional application No. 60/490,031, filed on Jul. 28, 2003.

(51) Int. Cl. A61K 33/14; A61K 31/522

(52) U.S. Cl. 424/680; 514/263.31

(57) ABSTRACT

Transporters are membrane proteins that translocate solutes across biological membranes. Active agents such as drugs, prodrugs, nutrients, nutraceuticals, and other bioactive substances are substrates for transporters. Some transporters require sodium to be co-transported with solute, in order to transport solute. This invention relates to a pharmaceutical formulation approach to enhance uptake of active agent by increasing the uptake of active agent by a sodium-dependent transporter, where sodium is fabricated with or co-administered with active agent. One example is the formulation of a dosage form containing the prodrug acyclovir valychenodeoxycholate, which targets the human apical sodium-dependent bile acid transporter, and sodium chloride to enhance active agent uptake from the gastrointestinal tract.

### CROSS-REFERENCE TO RELATED APPLICATIONS

This application claims the benefit of provisional patent application Ser. No 60/490,031, filed 2003 Jul. 25 by the University of Maryland, Baltimore and now assigned to the inventor.

### FEDERALLY SPONSORED RESEARCH

Not Applicable

### SEQUENCE LISTING OR PROGRAM

Not Applicable

### BACKGROUND OF THE INVENTION

This invention relates to a pharmaceutical formulation approach to increase active agent uptake by sodium-dependent transporters.

### BACKGROUND OF THE INVENTION—PRIOR ART

Transporters are membrane proteins that translocate solutes across biological membranes. Drugs, prodrugs, nutrients, nutraceuticals, and other bioactive substances are substrates for transporters. A substrate is a solute that is translocated by a transporter. Some transporters require sodium to be co-transported with substrate, in order to transport substrate. These transporters are denoted sodium-dependent transporters. Sodium-dependent transporters include members of (1-8):
the major facilitator superfamily (MFS) [Transporter Classification number 2.A.1]
the anion:cation symporter (ACS) family [TC no. 2.A.1.14]
the organic cation transporter (OCT) family [TC no. 2.A.1.19]
the vesicular neurotransmitter (VNT) family [TC no. 2.A.1.22]
the solute:sodium symporter (SSS) family [TC no. 2.A.21]
the neurotransmitter:sodium symporter (NSS) family [TC no. 2.A.22]
the dicarboxylate/amino acid:cation (Na+ or H+) symporter (DAACS) family [TC no. 2.A.23]
the citrate:cation symporter (CCS) family [TC no. 2.A.24]
the alanine or glycine:cation symporter (AGCS) family [TC no. 2.A.25]
the branched-chain amino acid:cation symporter (LIVCS) family [TC no. 2.A.26]
the glutamate:Na+ symporter (ESS) family [TC no. 2.A.27]
the bile acid:Na+ symporter (BASS) family [TC no. 2.A.28]
the nucleobase:cation symporter-2 (NCS2) family [TC no. 40]
the concentrative nucleoside transporter (CNT) family [TC no. 2.A.41]
the divalent anion:Na+ symporter (DASS) family [TC no. 2.A.47]
the reduced folate carrier (RFC) family [TC no. 2.A.48]
the phosphate:Na+ symporter (PNaS) family [TC no. 2.A.58]
and the malonate:Na+ symporter (MSS) family [TC no. 2.A.70].

The basic biology of transporters, including sodium-dependent transporters, is receiving attention since these proteins modulate the disposition of solutes in the body. Transporters are also now receiving attention as drug delivery targets. Valganciclovir [Valcyte] is a prodrug of the antiviral agent ganciclovir. Valganciclovir was designed a priori to employ a transporter to increase oral ganciclovir bioavailability. Valganciclovir targets hPepT1, the peptide transporter in the gut. PepT1 is not a sodium-dependent transporter. More recently, we have used the apical sodium-dependent bile acid transporter (ASBT) to increase the oral bioavailability of the poorly absorbed anti-viral acyclovir (9,10). ASBT is a sodium-dependent transporter. Human ASBT is denoted hASBT.

Although there has been at least some effort to exploit transporters as drug delivery targets, these limited efforts have focused on chemistry-based design considerations. For example, prodrugs of drugs have been designed to exploit transporters. There is no prior art that links sodium-dependent transporter-mediated uptake with a formulation approach that leverages the transporter's requirement of sodium.

### BACKGROUND OF INVENTION—OBJECTIVE AND ADVANTAGES

This invention relates to an approach to enhance uptake of active agent by increasing the uptake of active agent by a sodium-dependent transporter. Active agents can target sodium-dependent transporters in order to try to achieve improved uptake of the active agent. The formulation approach relies on co-administering the active agent with sodium. Preferably, the active agent is formulated into a dosage form with sodium.

FIG. 1 illustrates how this invention provides for improved uptake of active agent, and specifically the impact of sodium on the uptake of an acyclovir prodrug that utilizes the hASBT transporter, which is a sodium-dependent transporter. FIG. 1 shows sodium-enhanced uptake into COS-hASBT cells from prodrug acyclovir valylchenodeoxycholate. COS-hASBT cells express hASBT. Acyclovir valylchenodeoxycholate is a prodrug of acyclovir, where acyclovir valylchenodeoxycholate is a substrate for hASBT. Uptake from acyclovir valylchenodeoxycholate prodrug and uptake from acyclovir are shown as closed bar and open bar, respectively. Sodium did not enhance acyclovir uptake, as acyclovir is not a substrate for a sodium-dependent transporter. Uptake from acyclovir valylchenodeoxycholate prodrug was enhanced by sodium, as hASBT-mediated uptake of acyclovir valylchenodeoxycholate was stimulated by sodium. Error bars represent SEM's.

The effort to exploit sodium-dependent transporter to enhance active agent uptake can be improved though the described formulation approach. Active agents include drugs, prodrugs, nutrients, nutraceuticals, and other bioactive substances. Improved uptake includes increased permeability and penetration across a biological membrane. Improved uptake also can denote reduced variability in permeability and penetration across a biological membrane. Enhanced uptake denotes improved uptake. An example of improved uptake is increased oral absorption of active agent from the gastrointestinal tract in a human or animal, after oral administration. Oral administration entails the taking of active agent and sodium by mouth. Another example is increased exposure of a target organ or tissue to the active agent.

This invention entails the co-administration of active agent that is a substrate of a sodium-dependent transporter and sodium. Co-administration of active agent with sodium indicates that active agent and sodium are administered such that sodium is co-available with active agent to enhance uptake of active agent. This invention also entails the formulation of an active agent that is a substrate of a sodium-dependent transporter with sodium, in order to improve uptake of the active agent. Inclusion of sodium in a dosage form of active agent is preferable, as this approach is generally convenient.

In the context of formulation design, sodium can refer to any material or excipient that contains sodium. For example, sodium can refer to sodium chloride. The active agent itself can be a sodium salt. Containing sodium indicates the presence of sodium in any form.

This invention is applicable to a large number of sodium-dependent transporters, which are increasingly being leveraged as targets for improved active agent uptake. The table below lists example active agents, as well as a corresponding sodium-dependent nutrient transporter that the example agents are substrates for. For example, in the below table, acyclovir valychenodeoxycholate targets the human apical sodium-dependent bile acid transporter (hASBT), an example member of the BASS transporter family. Acyclovir valylchenodeoxycholate is a prodrug of the anti-viral acyclovir, and has already been shown enhance in vivo oral acyclovir bioavailability (9,10). As an example, this invention builds and expands upon this previous approach to employs hASBT to enhance oral bioavailability (10). Given an active agent's use of a sodium-dependent transporter to try to enhance uptake of the active agent, the current invention will facilitate favorable pharmacokinetics by administering the active agent with sodium. This formulation approach is applicable to the many other sodium-dependent transporters, including as-of-yet unknown transporters (11). This approach need not be limited to drugs and prodrugs, but can be applied to other active agents such as nutrients, bioactive substances, and nutraceuticals. This approach is not limited to the administration of active agents to humans, but includes application to all animals. This approach need not be limited to transporters at the intestinal level, but is also applicable to targets throughout the body (e.g. blood-brain barrier, liver, kidney, fetus) (12-32).

The examples in the table below are only examples. Other active agents using other transporters are also amendable to this formulation approach (e.g. monocarboxylates [such as thyroid hormone] using a sodium-dependent monocarboxylate transporter [such as MCT8]; iodine derivatives using the iodide transporter; dopamine using the creatine transporter; taurine using the taurine transporter), including active agents using sodium-dependent transporters that are unknown or undiscovered. In the table below, as is done in practice, the transporter is sometimes denoted by the gene that encodes for the transporter protein.

| Active agent | Example Transporter Family |
|---|---|
| acyclovir valychenodeoxycholate | bile acid:Na+ symporter (BASS) family |
| | (e.g. apical sodium-dependent bile acid |
| | transporter, Na+/taurocholate transport |
| | protein) [33, 34] |
| captopril deoxycholate | bile acid:Na+ symporter (BASS) family |
| | (e.g. apical sodium-dependent bile acid |
| | transporter, Na+/taurocholate transport |
| | protein) [33, 34] |
| biotin, pantothenate, lipoate | solute:sodium symporter (SSS) family (e.g. |
| | sodium-dependent multivitamin |
| | transporter) [35-40] |
| uridine, zidovudine, zaltidabine, cladribine, | concentrative nucleoside transporter (CNT) |
| cytarabine, gemcitabine, 5′deoxy-5- | family (e.g. CNT1) [41-46] |
| fluorouridine; other purine nucleosides and | |
| purine nucleoside analogs | |
| ribavirin, adenosine, cladribine, | concentrative nucleoside transporter (CNT) |
| didanosine; other pyridine nucleosides and | family (e.g. CNT2) [41-46] |
| pyridine nucleoside analogs | |
| 5-fluorouridine, floxuridine, zebularine, | concentrative nucleoside transporter (CNT) |
| gemcitabine, zalcitabine, didanoside; other | family (e.g. CNT3) [41-46] |
| purine nucleosides and purine nucleoside | |
| analogs | |
| alanine | dicarboxylate/amino acid:cation (Na+ or |
| | H+) symporter (DAACS) family (e.g. |
| | neutral amino acid:Na+ symporter; insulin- |
| | activated amino acid:Na+ symporter; |
| | broad-specificity amino acid:Na+ |
| | symporter); alanine or glycine:cation |
| | symporter (AGCS) family (e.g. alanine [or |
| | glycine]:Na+ symporter; Alanine:Na+ |
| | symporter) [47-54] |
| ascorbic acid, ascorbinic acid | dicarboxylate/amino acid:cation (Na+ or |
| | H+) symporter (DAACS) family (e.g. |
| | sodium-dependent vitamin C transporter 1, |
| | sodium-dependent vitamin C transporter 2, |
| | sodium-dependent vitamin C transporter 3) |
| | [55-58] |
| glucose, galactose, alpha-methyl- | sodium/glucose cotransporter (e.g. |
| glycopyranoside, inositol, proline, | SLC5A1, also known as SGLT1) [59] |
| pantothernate, iodine, urea, myoinositol; | |
| glucose derivatives such as 3-O-methyl- | |
| glucosed or quercetin glycosides | |
| glucose | sodium/glucose cotransporter (e.g. |
| | SLC5A2, also known as SGLT2) [59] |
| myo-inositol, glucose | sodium/glucose cotransporter (e.g. |
| | SLC5A4, also known as SGLT3) [59] |
| triethylamine, pyrilamine, quinidine, | organic cation transporter (e.g. OCTN2, |
| verapamil, carnitine, carnitine analogs, | also known as SLC22A5) |
| betaine, cephaloredine, choline, emetine, | |
| valproate | |
| HPO₄²⁻, phosphate derivatives | phosphate carrier system (e.g. NaPiIIb) |
| neutral amino acids, pregabalin | amino acid B⁰ carrier system |
| neutral amino acids | amino acid y⁺L carrier system (e.g. |
| | SLC7A7, SLC3A2) |
| neutral amino acids, glutamic acid, imino | amino acid A carrier system (e.g. SLC5A4) |
| acids | |
| cationic amino acids, neutral amino acids, | amino acid B^{O,+}carrier system (e.g. |
| pregabalin | SLC6A14) |
| beta-alanine, taurine | amino acid beta carrier system (e.g. |
| | SLC6A6) |
| aspartic acid, glutamic acid, glutamic acid- | amino acid X_{AG-}carrier system (e.g. |
| 1a, aspartic acid-3 | SLC1A5) |
| alanine, serine, cystine, glycine, threonine, | amino acid Asc carrier system (e.g. |
| alpha-aminobutyric acid, beta-alanine, D- | SLC7A10, SLC3A2) |
| serine | |

### SUMMARY

Some active agents, such as some drugs, prodrugs, nutrients, nutraceuticals, and other bioactive substances, are substrates for sodium-dependent transporters. This invention relates to a pharmaceutical formulation approach to enhance uptake of active agent by increasing the uptake of active agent by a sodium-dependent transporter, where sodium is fabricated with or co-administered with active agent. One example is the formulation of a dosage form containing the prodrug acyclovir valychenodeoxycholate, which targets the human apical sodium-dependent bile acid transporter, and sodium chloride to enhance active agent uptake from the gastrointestinal tract.

### DRAWINGS

FIG. 1 highlights the ability of sodium to enhance active agent uptake.

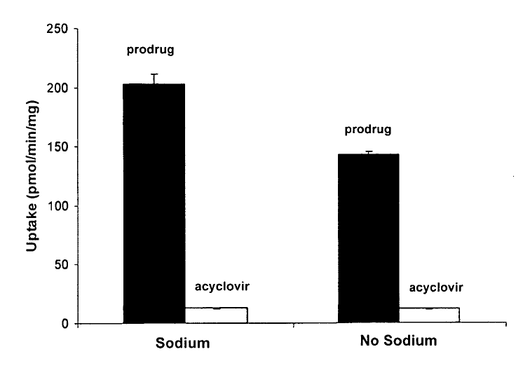

### DETAILED DESCRIPTION

Preferred Embodiment

This formulation approach relies on sodium-dependent transporter-mediated uptake, but is not limited to active agents with only one therapeutic category. Enhanced oral absorption is the primary area of potential application. The use of a material with a relatively high sodium composition on a weight basis, especially sodium chloride, is preferred. Preferably, co-administration of sodium will be achieved by formulating active agent with sodium in a dosage form, where at least 0.5 milliequivalent of sodium is present.

The following materials were use. Acyclovir valychenodeoxycholate was synthesized in Dr. Polli's laboratory using previously described methods (10). Captopril deoxycholate was similarly synthesized in Dr. Polli's laboratory. Biotin, zidovudine, ribavirin, alanine, and ascorbic acid were obtained from Sigma (St. Louis, Mo.). Sodium chloride and sodium citrate tribasic dihydrate were obtained from Sigma (St. Louis, Mo.). Microcrystalline cellulose (Avicel PH101) and croscarmellose sodium (Ac-Di-Sol) were obtained from FMC Biopolymer (Newark, Del.). Magnesium stearate, sodium phosphate monobasic granular, sodium citrate tribasic dihydrate granular, and sodium starch glycolate were obtained from Spectrum (Gardina, Calif.). Sodium phosphate dibasic anhydrous was obtained from EM Industries (Gibbstown, N.J.). Silicified microcrystalline cellulose (Prosolv SMCC90) was obtained from Mendell (Patterson, N.J.). Crospovidone (Polyplasdone XL-10) was obtained from ISP Technologies Inc. (Wayne, N.J.). Lactose anhydrous was obtained from Quest International (Hoffman Estates, Ill.). Corn starch was obtained from Roquette America Inc. (Keokuk, Iowa). Dicalcium phosphate anhydrous was obtained from Rhone-Poulenc (Cranbury, N.J.). Carboxymethylcellulose sodium was obtained from Sigma (St. Louis, Mo.).

For a number of active agents, tablets containing an active agent that is a substrate for a sodium-dependent transporter and containing sodium were fabricated. For each active agent, tablets were characterized in terms of their hardness and in vitro dosage form release properties, specifically in vitro disintegration and in vitro dissolution of sodium. Using a compendial dissolution test, each dosage form delivered sodium to the dissolution medium, from where active agent is taken up by one (or more) sodium-dependent transporters.

Tablets were formulation to containing sodium and active agent that was a substrate for a sodium-dependent transporter. Formulations A-K were manufactured. Capsules, powders, solutions, suspensions, and other dosage forms are also possible (60). Co-administration of a formulation of active agent and a formulation of sodium is also possible. Each formulation A-K contains sodium (i.e. a material containing sodium, typically a sodium salt). Sodium-possessing formulation components were: sodium chloride, croscarmellose sodium, sodium phosphate dibasic anhydrous, sodium citrate tribasic dihydrate, sodium phosphate monobasic granular, sodium citrate tribasic dihydrate granular, sodium starch glycolate, and carboxymethylcellulose sodium. Many other sodium-possessing excipients are suitable as formulation components to provide sodium, and can also provide formulation benefit as fillers, binder, buffers, disintegrants, and other roles known in the art (61-63). While the current examples indicate the fabrication of a formulation that includes a sodium-possessing substance and an active agent, the describe approach can also be applied when the sodium-possessing substance and the active agent are not formulated as one dosage form, but are co-administered. However, the inclusion of sodium in a dosage form of active agent is preferable, as this approach is generally convenient. Each formulation also contained an active agent that is a substrate for a transporter that co-transports sodium ion. In these examples, active agents were: acyclovir valychenodeoxycholate, captopril deoxycholate, biotin, zidovudine, ribaviran, alanine, and ascorbic acid. Sodium salts of the active also can provide sodium. Formulations and other administration regimens can also include more than one active agent.

In the examples below, most formulations also employed materials that were neither the active agent nor sodium-possessing (60). Examples include microcrystalline cellulose, magnesium stearate, silicified microcrystalline cellulose, crospovidone, lactose anhydrous, corm starch, and dicalcium phosphate anhydrous. Such materials are well-known to facilitate dosage form fabrication and/or dosage for performance.

For each formulation, six tablets were subjected to tablet hardness testing using a Key hardness tester [model HT-300] (Key International, Inc., Elizabeth, N.J.). Values were measure in units of kilopond (KP) and converted to units of Newton (N).

For each formulation, tablet disintegration testing and tablet dissolution testing were performed to assess the ability of the tablet to provide sodium ion into the surrounding medium. Six tablets were evaluated in the disintegration test. Either six or twelve tablets were evaluated in the dissolution test. The disintegration apparatus conformed to USP compendial specifications. The disintegration apparatus components [model Vanderkamp] were manufactured by Van Kel Industries (Edison, N.J.): the basket-rack assembly, motor, water heater, and water bath. Disintegration was performed at 30 cycles/min using 900 mL water at 37° C. in a 1 L flat-bottom flask.

The dissolution apparatus conformed to USP compendial specifications. The apparatus was manufactured [model VK 700] by Van Kel Industries (Edison, N.J.), and also employed a water heater [model VK 750D] (Van Kel Industries, Edison, N.J.). Dissolution was performed with paddle using either 900 mL water at 37° C. or 900 mL water that had been adjusted to pH 1.5 at 37° C. Water was employed in evaluating Formulations A, B, C, G, H, I, J, and K. Water adjusted to pH 1.5 was employed in evaluating Formulations D, E, and F. A single sample was taken from each vessel at either 5, 10, or 30 min. Sodium was quantified using a Jenway flame photometer [model PFP7] (Jenway, Princeton, N.J.). The standard curve was linear (r²=0.997); standards were fitted with acceptable accuracy (<2% error).

Results were analyzed by student's t-test or by ANOVA with post hoc analysis, using SPSS version 10 (SPSS, Chicago, Ill.). A p-value less than 0.05 was considered significant. SEM's of ratios were calculated by the delta method.

Formulations A-K each resulted in tablets that were white, round, and flat-faced. Other tablet tooling can be employed to provide other tablet shapes. Other excipients or formulations processes (e.g. coating) can be used to yield other tablet appearance. For each formulation, tablet hardness, disintegration, and dissolution attributes are listed below. Disintegration and sodium ion dissolution data reflect availability of sodium ion from the formulation.

### Formulation A

Tablets were fabricated from a powder mixture of acyclovir valychenodeoxycholate (100 mg/tablet), sodium chloride (250 mg/tablet), and microcrystalline cellulose (150 mg/tablet). Individual components were weighed out for 20 tablets and combined in a mortar to yield a uniform mixture. 500 mg of powder mixture was compressed on a Carver laboratory press [model 4687] (Fred S. Carver Inc., Menomee Falls, Wis.) using tablet tooling. The compact was compressed to 500 psi for 60 sec.

Tablet Hardness of Formulation A

| Tablet | Tablet Hardness (N) | |
|---|---|---|
| 1 | 28.4 | |
| 2 | 31.4 | |
| 3 | 32.4 | |
| 4 | 28.4 | |
| 5 | 30.4 | |
| 6 | 32.4 | |
| Mean (±SE) | 30.6 | (±0.8) |

Table Disintegration of Formulation A

| Tablet | Disintegration Time (sec) |
|---|---|
| 1 | 4-5 |
| 2 | 4-5 |
| 3 | 4-5 |
| 4 | 4-5 |
| 5 | 4-5 |
| 6 | 4-5 |
| Mean | 4-5 |

Tablet Dissolution of Formulation A

| | Percent Sodium Ion Dissolved at Five | |
|---|---|---|
| Tablet | Minutes | |
| 1 | 112.5 | |
| 2 | 99.2 | |
| 3 | 106.9 | |
| 4 | 102.1 | |
| 5 | 96.9 | |
| 6 | 103.5 | |
| 7 | 99.7 | |
| 8 | 103.8 | |
| 9 | 101.4 | |
| 10 | 92.8 | |
| 11 | 109.0 | |
| 12 | 103.1 | |
| Mean (±SE) | 102.6 | (±1.6) |

### Formulation B

Tablets were fabricated from a powder mixture of acyclovir valychenodeoxycholate (200 mg/tablet) and sodium chloride (300 mg/tablet). Individual components were weighed out for 20 tablets and combined in a mortar to yield a uniform mixture. 500 mg of powder mixture was compressed on a Carver laboratory press. The compact was compressed to 1000 psi for 60 sec.

Tablet Hardness of Formulation B

| Tablet | Tablet Hardness (N) | |
|---|---|---|
| 1 | 39.2 | |
| 2 | 36.3 | |
| 3 | 40.2 | |
| 4 | 41.2 | |
| 5 | 37.3 | |
| 6 | 38.2 | |
| Mean (±SE) | 38.7 | (±0.7) |

Table Disintegration of Formulation B

| Tablet | Disintegration Time (sec) |
|---|---|
| 1 | 9 |
| 2 | 9 |
| 3 | 7 |
| 4 | 7 |
| 5 | 6 |
| 6 | 6 |
| Mean | 7.4 (±0.5) |

Tablet Dissolution of Formulation B

| | Percent Sodium Ion Dissolved at Five | |
|---|---|---|
| Tablet | Minutes | |
| 1 | 102.0 | |
| 2 | 101.1 | |
| 3 | 102.1 | |
| 4 | 95.1 | |
| 5 | 101.0 | |
| 6 | 107.9 | |
| Mean (±SE) | 101.6 | (±1.5) |

### Formulation C

Tablets were fabricated from a powder mixture of acyclovir valychenodeoxycholate (200 mg/tablet), sodium chloride (300 mg/tablet), croscamellose sodium (20 mg/tablet), and magnesium stearate (4 mg/tablet). Individual components were weighed out for 20 tablets and combined in a mortar to yield a uniform mixture. 524 mg of powder mixture was compressed on a Carver laboratory press. The compact was compressed to 1000 psi for 60 sec.

Tablet Hardness of Formulation C

| Tablet | Tablet Hardness (N) | |
|---|---|---|
| 1 | 33.3 | |
| 2 | 33.3 | |
| 3 | 31.4 | |
| 4 | 37.3 | |
| 5 | 34.3 | |
| 6 | 35.3 | |
| Mean (±SE) | 30.6 | (±0.7) |

Table Disintegration of Formulation C

| Tablet | Disintegration Time (sec) |
|---|---|
| 1 | <3 |
| 2 | <3 |
| 3 | <3 |
| 4 | <3 |
| 5 | <3 |
| 6 | <3 |
| Mean | <3 |

Tablet Dissolution of Formulation C

| | Percent Sodium Ion Dissolved at Five | |
|---|---|---|
| Tablet | Minutes | |
| 1 | 101.6 | |
| 2 | 99.5 | |
| 3 | 98.5 | |
| 4 | 94.3 | |
| 5 | 98.6 | |
| 6 | 93.9 | |
| Mean (±SE) | 97.7 | (±1.1) |

### Formulation D

Tablets were fabricated from a powder mixture of acyclovir valychenodeoxycholate (200 mg/tablet) and sodium phosphate dibasic anhydrous (200 mg/tablet). Individual components were weighed out for 20 tablets and combined in a mortar to yield a uniform mixture. 400 mg of powder mixture was compressed on a Carver laboratory press. The compact was compressed to 1000 psi for 60 sec.

Tablet Hardness of Formulation D

| Tablet | Tablet Hardness (N) | |
|---|---|---|
| 1 | 34.3 | |
| 2 | 33.3 | |
| 3 | 33.3 | |
| 4 | 35.3 | |
| 5 | 34.3 | |
| 6 | 36.3 | |
| Mean (±SE) | 34.5 | (±0.4) |

Table Disintegration of Formulation D

| Tablet | Disintegration Time (sec) | |
|---|---|---|
| 1 | 130 | |
| 2 | 160 | |
| 3 | 160 | |
| 4 | 160 | |
| 5 | 160 | |
| 6 | 180 | |
| Mean | 158 | (±6) |

Tablet Dissolution of Formulation D

| | Percent Sodium Ion Dissolved at Thirty |
|---|---|
| Tablet | Minutes |
| 1 | 106.0 |
| 2 | 94.2 |
| 3 | 106.5 |
| 4 | 102.0 |
| 5 | 105.0 |
| 6 | 103.7 |
| Mean (±SE) | (±1.9) |

### Formulation E

Tablets were fabricated from a powder mixture of acyclovir valychenodeoxycholate (200 mg/tablet), sodium phosphate dibasic anhydrous (200 mg/tablet), silicified microcrystalline cellulose (100 mg/tablet), crospovidone (20 mg/tablet), and magnesium stearate (4 mg/tablet). Individual components were weighed out for 20 tablets and combined in a mortar to yield a uniform mixture. 524 mg of powder mixture was compressed on a Carver laboratory press. The compact was compressed to 1000 psi for 60 sec.

Tablet Hardness of Formulation E

| Tablet | Tablet Hardness (N) | |
|---|---|---|
| 1 | 37.3 | |
| 2 | 65.7 | |
| 3 | 41.2 | |
| 4 | 46.1 | |
| 5 | 47.1 | |
| 6 | 48.1 | |
| Mean (±SE) | 47.6 | (±4.6) |

Table Disintegration of Formulation E

| Tablet | Disintegration Time (sec) | |
|---|---|---|
| 1 | 130 | |
| 2 | 150 | |
| 3 | 160 | |
| 4 | 160 | |
| 5 | 170 | |
| 6 | 210 | |
| Mean | 163 | (±10) |

Tablet Dissolution of Formulation E

| | Percent Sodium Ion Dissolved at Ten | |
|---|---|---|
| Tablet | Minutes | |
| 1 | 103.4 | |
| 2 | 100.5 | |
| 3 | 104.5 | |
| 4 | 105.9 | |
| 5 | 92.5 | |
| 6 | 98.9 | |
| Mean (±SE) | 101.0 | (±1.8) |

### Formulation F

Tablets were fabricated from a powder mixture of captopril deoxycholate (25 mg/tablet), sodium citrate tribasic dihydrate (250 mg/tablet), microcrystalline cellulose (150 mg/tablet), lactose anhydrous (50 mg/tablet), croscarmellose sodium (30 mg/tablet), and magnesium stearate (3 mg/tablet). Individual components were weighed out for 20 tablets and combined in a mortar to yield a uniform mixture. 508 mg of powder mixture was compressed on a Carver laboratory press. The compact was compressed to 1000 psi for 60 sec.

Tablet Hardness of Formulation F

| Tablet | Tablet Hardness (N) | |
|---|---|---|
| 1 | 56.9 | |
| 2 | 55.9 | |
| 3 | 50.0 | |
| 4 | 57.9 | |
| 5 | 60.8 | |
| 6 | 53.9 | |
| Mean(±SE) | 55.9 | (±1.4) |

Table Disintegration of Formulation F

| Tablet | Disintegration Time (sec) | |
|---|---|---|
| 1 | 80 | |
| 2 | 85 | |
| 3 | 85 | |
| 4 | 85 | |
| 5 | 95 | |
| 6 | 95 | |
| Mean | 88 | (±2) |

Tablet Dissolution of Formulation F

| | Percent Sodium Ion Dissolved at Five | |
|---|---|---|
| Tablet | Minutes | |
| 1 | 98.3 | |
| 2 | 93.0 | |
| 3 | 97.4 | |
| 4 | 96.3 | |
| 5 | 98.4 | |
| 6 | 96.6 | |
| Mean (±SE) | 96.7 | (±0.7) |

### Formulation G

Tablets were fabricated from a powder mixture of biotin (30 microgram/tablet), sodium phosphate monobasic granular (100 mg/tablet), microcrystalline cellulose (200 mg/tablet), and corn starch (20 mg/tablet). Individual components were weighed out for 20 tablets and combined in a mortar to yield a uniform mixture. 320.030 mg of powder mixture was compressed on a Carver laboratory press. The compact was compressed to 1000 psi for 60 sec.

Tablet Hardness of Formulation G

| Tablet | Tablet Hardness (N) | |
|---|---|---|
| 1 | 60.8 | |
| 2 | 63.7 | |
| 3 | 65.7 | |
| 4 | 53.0 | |
| 5 | 57.9 | |
| 6 | 60.8 | |
| Mean (±SE) | 60.3 | (±1.7) |

Table Disintegration of Formulation G

| Tablet | Disintegration Time (sec) | |
|---|---|---|
| 1 | 62 | |
| 2 | 65 | |
| 3 | 75 | |
| 4 | 75 | |
| 5 | 80 | |
| 6 | 90 | |
| Mean | 75 | (±4) |

Tablet Dissolution of Formulation G

| | Percent Sodium Ion Dissolved at Ten | |
|---|---|---|
| Tablet | Minutes | |
| 1 | 111.2 | |
| 2 | 108.2 | |
| 3 | 93.0 | |
| 4 | 105.5 | |
| 5 | 111.4 | |
| 6 | 106.3 | |
| Mean (±SE) | 105.9 | (±2.5) |

### Formulation H

Tablets were fabricated from a powder mixture of zidovudine (100 mg/tablet), sodium citrate tribasic dihydrate granular (100 mg/tablet), silicified microcrystalline cellulose (200 mg/tablet), lactose anhydrous (50 mg/tablet), and sodium starch glycolate (25 mg/tablet). Individual components were weighed out for 20 tablets and combined in a mortar to yield a uniform mixture. 475 mg of powder mixture was compressed on a Carver laboratory press. The compact was compressed to 1000 psi for 60 sec.

Tablet Hardness of Formulation H

| Tablet | Tablet Hardness (N) | |
|---|---|---|
| 1 | 46.1 | |
| 2 | 41.2 | |
| 3 | 40.2 | |
| 4 | 47.1 | |
| 5 | 52.0 | |
| 6 | 50.0 | |
| Mean (±SE) | 46.1 | (±1.7) |

Table Disintegration of Formulation H

| Tablet | Disintegration Time (sec) | |
|---|---|---|
| 1 | 42 | |
| 2 | 47 | |
| 3 | 52 | |
| 4 | 53 | |
| 5 | 61 | |
| 6 | 63 | |
| Mean | 53 | (±3) |

Tablet Dissolution of Formulation H

| | Percent Sodium Ion Dissolved at Five | |
|---|---|---|
| Tablet | Minutes | |
| 1 | 104.3 | |
| 2 | 106.2 | |
| 3 | 103.5 | |
| 4 | 109.4 | |
| 5 | 99.0 | |
| 6 | 109.2 | |
| Mean (±SE) | 105.3 | (±1.5) |

### Formulation I

Tablets were fabricated from a powder mixture of ribavirin (200 mg/tablet), sodium chloride (150 mg/tablet), microcrystalline cellulose (200 mg/tablet), lactose anhydrous (25 mg/tablet), and magnesium stearate (3 mg/tablet). Individual components were weighed out for 20 tablets and combined in a mortar to yield a uniform mixture. 478 mg of powder mixture was compressed on a Carver laboratory press. The compact was compressed to 1000 psi for 60 sec.

Tablet Hardness of Formulation I

| Tablet | Tablet Hardness (N) | |
|---|---|---|
| 1 | 41.2 | |
| 2 | 46.1 | |
| 3 | 47.1 | |
| 4 | 38.2 | |
| 5 | 40.2 | |
| 6 | 38.2 | |
| Mean (±SE) | 41.8 | (±1.4) |

Table Disintegration of Formulation I

| Tablet | Disintegration Time (sec) |
|---|---|
| 1 | <5 |
| 2 | <5 |
| 3 | <5 |
| 4 | <5 |
| 5 | <5 |
| 6 | <5 |
| Mean | <5 |

Tablet Dissolution of Formulation I

| | Percent Sodium Ion Dissolved at Five | |
|---|---|---|
| Tablet | Minutes | |
| 1 | 106.2 | |
| 2 | 103.2 | |
| 3 | 100.5 | |
| 4 | 106.4 | |
| 5 | 101.3 | |
| 6 | 95.0 | |
| Mean (±SE) | 102.1 | (±1.6) |

### Formulation J

Tablets were fabricated from a powder mixture of alanine (100 mg/tablet), sodium chloride (200 mg/tablet), sodium phosphate monobasic granular (50 mg/tablet), silicified microcrystalline cellulose (100 mg/tablet), and croscarmellose sodium (40 mg/tablet). Individual components were weighed out for 20 tablets and combined in a mortar to yield a uniform mixture. 490 mg of powder mixture was compressed on a Carver laboratory press. The compact was compressed to 1000 psi for 60 sec.

Tablet Hardness of Formulation J

| Tablet | Tablet Hardness (N) | |
|---|---|---|
| 1 | 41.2 | |
| 2 | 50.0 | |
| 3 | 37.3 | |
| 4 | 38.2 | |
| 5 | 44.1 | |
| 6 | 50.0 | |
| Mean (±SE) | 43.5 | (±2.1) |

Table Disintegration of Formulation J

| Tablet | Disintegration Time (sec) |
|---|---|
| 1 | <5 |
| 2 | <5 |
| 3 | <5 |
| 4 | <5 |
| 5 | <5 |
| 6 | <5 |
| Mean | <5 |

Tablet Dissolution of Formulation J

| | Percent Sodium Ion Dissolved at Five | |
|---|---|---|
| Tablet | Minutes | |
| 1 | 105.4 | |
| 2 | 105.2 | |
| 3 | 102.2 | |
| 4 | 99.5 | |
| 5 | 98.0 | |
| 6 | 100.3 | |
| Mean (±SE) | 101.8 | (±1.1) |

### Formulation K

Tablets were fabricated from a powder mixture of ascorbic acid (100 mg/tablet), sodium chloride (150 mg/tablet), dicarcium phosphate anhydrous (25 mg/tablet), silicified microcrystalline cellulose (100 mg/tablet), carboxymethylcellulose sodium (25 mg/tablet), and croscarmellose sodium (25 mg/tablet). Individual components were weighed out for 20 tablets and combined in a mortar to yield a uniform mixture. 425 mg of powder mixture was compressed on a Carver laboratory press. The compact was compressed to 1000 psi for 60 sec.

Tablet Hardness of Formulation K

| Tablet | Tablet Hardness (N) | |
|---|---|---|
| 1 | 44.1 | |
| 2 | 41.2 | |
| 3 | 46.1 | |
| 4 | 50.0 | |
| 5 | 40.2 | |
| 6 | 42.2 | |
| Mean (±SE) | 44.0 | (±1.4) |

Table Disintegration of Formulation K

| Tablet | Disintegration Time (sec) | |
|---|---|---|
| 1 | 5 | |
| 2 | 5 | |
| 3 | 5 | |
| 4 | 7 | |
| 5 | 7 | |
| 6 | 7 | |
| Mean | 6 | (±0.4) |

Tablet Dissolution of Formulation K

| | Percent Sodium Ion Dissolved at Five | |
|---|---|---|
| Tablet | Minutes | |
| 1 | 101.0 | |
| 2 | 107.2 | |
| 3 | 104.2 | |
| 4 | 101.5 | |
| 5 | 99.0 | |
| 6 | 102.3 | |
| Mean (±SE) | 102.5 | (±1.1) |

The above formulations serve as examples. This approach provides for the delivery of sodium ion along with the delivery of an active agent that targets for a sodium-dependent transporter, in order to enhance uptake of the active agent. Targets indicates that the active agent is a substrate for a sodium-dependent transporter. In Formulation A, the solute is acyclovir valylchenodeoxycholate, a prodrug of acyclovir and which targets the BASS family, including the human apical sodium-dependent bile acid transporter (hASBT). A tablet was designed and manufactured to include both the prodrug and sodium chloride, as source of sodium ion. Other dosage forms (e.g. capsules, waffer) and other regimens (e.g. co-administration of two dosage forms, one containing the active agent and the other providing sodium) are possible. Other sources of sodium (e.g. sodium citrate, sodium phosphate) can be included in addition to sodium chloride and/or in place of sodium chloride. Sodium species are not typically designed into tablet and capsule formulations. The tablet was designed to release sodium in an immediate release fashion. Other release profiles (e.g. sustained release, delayed release) are also possible.

Disintegration and dissolution are common in vitro tools to predict in vivo performance. Disintegration and dissolution are compendial tests. Disintegration and dissolution data indicate the availability of sodium ion from the dosage form into the medium from which prodrug is taken up by the transporter. Cell uptake studies indicate sodium to enhance hASBT uptake of prodrug. This prodrug approach itself has been shown in rats to enhance acyclovir oral bioavailability.

This formulation approach is applicable to other sodium-dependent transporters in the gastrointestinal tract and throughout the body. Hence, in addition to improved oral absorption from the gut, this approach can improve the uptake of active agents into other tissues and organs (e.g. uptake to brain).

All references cited herein are incorporated by reference in their entirety.

References:

1. Busch W. Saier M H Jr. The transporter classification (TC) system, 2002. Critical Reviews in Biochemistry & Molecular Biology. 37(5):287-337, 2002.
2. Saier M H Jr. A functional-phylogenetic classification system for transmembrane solute transporters. Microbiology & Molecular Biology Reviews. 64(2):354-411, 2000.
3. Jung H. The sodium/substrate symporter family: structural and functional features. FEBS Letters. 529(1):73-7, 2002.
4. M. H. Saier Jr. Tracing pathways of transport protein evolution. Molecular Microbiology 48:1145-56, 2003.
5. E. Y. Zhang, M. A. Phelps, C. Cheng, S. Ekins, and P. W. Swaan. Modeling of active transport systems. Advanced Drug Delivery Reviews 54(3):329-54, 2002.
6. B. Steffansen, C. U. Nielsen, B. Brodin, A. H. Eriksson, R. Andersen, and S. Frokjaer. Intestinal solute carriers: an overview of trends and strategies for improving oral drug absorption. European Journal of Pharmaceutical Sciences 21: 3-16, 2004.
7. http://www-biology.ucsd.edu/˜msaier/transport/
8. http://www.gene.ucl.ac.uk/
9. J. E. Polli, K. A. Lentz, D. Y. Maeda and A. Coop: Bile Acid Prodrug Conjugates for Enhanced Intestinal Uptake and Bioavailability. Provisonal Application No. 60/195,854. (PCT/US01/11327).
10. S. Tolle-Sander, K. A. Lentz, D. Y. Maeda, A. Coop, A., and J. E. Polli. Increased acyclovir oral bioavailability via a bile acid conjugate. Molecular Pharmaceutics 1:40-48, 2004.
11. P. Anderle, Y. Huang, and W. Sadee. Intestinal membrane transport of drugs and nutrients: genomics of membrane transporters using expression microarrays. European Journal of Pharmaceutical Sciences 21: 17-24, 2004.
12. Murer H. Biber J. Membrane permeability. Epithelial transport proteins: physiology and pathophysiology. Current Opinion in Cell Biology. 10(4):429-34, 1998.
13. Tamai I. Tsuji A. Transporter-mediated permeation of drugs across the blood-brain barrier. Journal of Pharmaceutical Sciences. 89(11):1371-88, 2000.
14. Ganapathy V. Prasad P D. Ganapathy M E. Leibach F H. Placental transporters relevant to drug distribution across the maternal-fetal interface. Journal of Pharmacology & Experimental Therapeutics. 294(2):413-20, 2000.
15. Kato Y. Suzuki H. Sugiyama Y. Toxicological implications of hepatobiliary transporters. Toxicology. 181-182:287-90, 2002.
16. Ayrton A. Morgan P. Role of transport proteins in drug absorption, distribution and excretion. Xenobiotica. 31(8-9):469-97, 2001.
17. Dresser M J. Leabman M K. Giacomini K M. Transporters involved in the elimination of drugs in the kidney: organic anion transporters and organic cation transporters. Journal of Pharmaceutical Sciences. 90(4):397-421, 2001.
18. Inui K I. Masuda S. Saito H. Cellular and molecular aspects of drug transport in the kidney. Kidney International. 58(3):944-58, 2000.
19. Ohashi R. Tamai I. Yabuuchi H. Nezu J I. Oku A. Sai Y. Shimane M. Tsuji A. Na(+)-dependent carnitine transport by organic cation transporter (OCTN2): its pharmacological and toxicological relevance. Journal of Pharmacology & Experimental Therapeutics. 291(2):778-84, 1999.
20. Tamai, I.; Ohashi, R.; Nezu, J.; Yabuuchi, H.; Oku, A.; Shimane, M.; Sai, Y.; Tsuji, A.: Molecular and functional identification of sodium ion-dependent, high affinity human carnitine transporter OCTN2. J. Biol. Chem. 273: 20378-20382, 1998.
21. Tamai I. Ohashi R. Nezu J I. Sai Y. Kobayashi D. Oku A. Shimane M. Tsuji A. Molecular and functional characterization of organic cation/carnitine transporter family in mice. Journal of Biological Chemistry. 275(51):40064-72, 2000.
22. Lahjouji K. Mitchell G A. Qureshi I A. Carnitine transport by organic cation transporters and systemic carnitine deficiency. Molecular Genetics & Metabolism. 73(4):287-97, 2001.
23. Ganapathy, M. E., W. Huang, D. P. Rajan, A. L. Carter, M. Sugawara, K. Iseki, F. H. Leibach, and V. Ganapathy. (2000). Beta-lactam antibiotics as substrates for OCTN2, an organic cation/carnitine transporter. J. Biol. Chem. 275: 1699-1707.
24. Wood I S. Trayhurn P. Glucose transporters (GLUT and SGLT): expanded families of sugar transport proteins. British Journal of Nutrition. 89(1):3-9, 2003.
25. Koistinen H A. Zierath J R. Regulation of glucose transport in human skeletal muscle. Annals of Medicine. 34(6):410-8, 2002.
26. Turk E. Wright E M. Membrane topology motifs in the SGLT cotransporter family. Journal of Membrane Biology. 159(1): 1-20, 1997.
27. Cho J Y. A transporter gene (sodium iodide symporter) for dual purposes in gene therapy: imaging and therapy. Current Gene Therapy. 2(4):393-402, 2002.
28. Chung J K. Sodium iodide symporter: its role in nuclear medicine. Journal of Nuclear Medicine. 43(9):1188-200, 2002.
29. Riedel C. Dohan O. De la Vieja A. Ginter C S. Carrasco N. Journey of the iodide transporter NIS: from its molecular identification to its clinical role in cancer. Trends in Biochemical Sciences. 26(8):490-6, 2001.
30. Uchida, S.; Kwon, H. M.; Yamauchi, A.; Preston, A. S.; Marumo, F.; Handler, J. S.: Molecular cloning of the cDNA for an MDCK cell Na(+)− and Cl(−)-dependent taurine transporter that is regulated by hypertonicity. Proc. Nat. Acad. Sci. 89: 8230-8234, 1992.
31. A. Laakso and J. Hietala. PET studies of brain monoamine transporters. Current Pharmaceutical Design 6:1611-23, 2000.
32. Zhou X. Hvorup R N. Saier M H Jr. An automated program to screen databases for members of protein families. Journal of Molecular Microbiology and Biotechnology 5:7-10, 2003.
33. Weinman S A. Carruth M W. Dawson P A. Bile acid uptake via the human apical sodium-bile acid cotransporter is electrogenic. Journal of Biological Chemistry. 273(52):34691-5, 1998.
34. Meier P J. Stieger B. Bile salt transporters. Annual Review of Physiology. 64:635-61, 2002.
35. Prasad, P. D.; Wang, H.; Kekuda, R.; Fujita, T.; Fei, Y.-J.; Devoe, L. D.; Leibach, F. H.; Ganapathy, V. Cloning and functional expression of a cDNA encoding a mammalian sodium-dependent vitamin transporter mediating the uptake of pantothenate, biotin, and lipoate. J. Biol. Chem. 273: 7501-7506, 1998.
36. Balamurugan K. Ortiz A. Said H M. Biotin uptake by human intestinal and liver epithelial cells: role of the SMVT system. American Journal of Physiology—Gastrointestinal & Liver Physiology. 285(1):G73-7, 2003.
37. Minko T. Paranjpe P V. Qiu B. Lalloo A. Won R. Stein S. Sinko P J. Enhancing the anticancer efficacy of camptothecin using biotinylated poly(ethylene glycol) conjugates in sensitive and multidrug-resistant human ovarian carcinoma cells. Chemotherapy & Pharmacology. 50(2):143-50, 2002.
38. Ramanathan S. Pooyan S. Stein S. Prasad P D. Wang J. Leibowitz M J. Ganapathy V. Sinko P J. Targeting the sodium-dependent multivitamin transporter (SMVT) for improving the oral absorption properties of a retro-inverso Tat nonapeptide. Pharmaceutical Research. 18(7):950-6, 2001.
39. Prasad P D. Wang H. Huang W. Fei Y J. Leibach F H. Devoe L D. Ganapathy V. Molecular and functional characterization of the intestinal Na+-dependent multivitamin transporter. Archives of Biochemistry & Biophysics. 366(1):95-106, 1999.
40. Wang H. Huang W. Fei Y J. Xia H. Yang-Feng T L. Leibach F H. Devoe L D. Ganapathy V. Prasad P D. Human placental Na+-dependent multivitamin transporter. Cloning, functional expression, gene structure, and chromosomal localization. Journal of Biological Chemistry. 274(21):14875-83, 1999.
41. Baldwin SA. Mackey J R. Cass C E. Young J D. Nucleoside transporters: molecular biology and implications for therapeutic development. Molecular Medicine Today. 5(5):216-24, 1999.
42. Pastor-Anglada M. Felipe A. Casado F J. Transport and mode of action of nucleoside derivatives used in chemical and antiviral therapies. Trends in Pharmacological Sciences. 19(10):424-30, 1998.
43. Lai Y. Bakken A H. Unadkat J D. Simultaneous expression of hCNT1-CFP and hENT1-YFP in Madin-Darby canine kidney cells. Localization and vectorial transport studies. Journal of Biological Chemistry. 277(40):37711-7, 2002.
44. Patil S D. Ngo L Y. Unadkat J D. Structure-inhibitory profiles of nucleosides for the human intestinal N1 and N2 Na+-nucleoside transporters. Cancer Chemotherapy & Pharmacology. 46(5):394-402, 2000.
45. Lum P Y. Ngo L Y. Bakken A H. Unadkat J D. Human intestinal es nucleoside transporter: molecular characterization and nucleoside inhibitory profiles. Cancer Chemotherapy & Pharmacology. 45(4):273-8, 2000.
46. Patil S D. Ngo L Y. Glue P. Unadkat J D. Intestinal absorption of ribavirin is preferentially mediated by the Na+-nucleoside purine (N1) transporter. Pharmaceutical Research. 15(6):950-2, 1998.
47. Kanai Y. Endou H. Heterodimeric amino acid transporters: molecular biology and pathological and pharmacological relevance. Current Drug Metabolism. 2(4):339-54, 2001.
48. Yang C. Tirucherai G S. Mitra A K. Prodrug based optimal drug delivery via membrane transporter/receptor. Expert Opinion on Biological Therapy. 1(2):159-75, 2001.
49. Jansson T. Amino acid transporters in the human placenta. Pediatric Research. 49(2):141-7, 2001.
50. Kanai Y. Segawa H. Chairoungdua A. Kim J Y. Kim D K. Matsuo H. Cha S H. Endou H. Amino acid transporters: molecular structure and physiological roles. Nephrology Dialysis Transplantation. 15 Suppl 6:9-10, 2000.
51. Smith Q R. Transport of glutamate and other amino acids at the blood-brain barrier. Journal of Nutrition. 130(4S Suppl): 1016S-22S, 2000.
52. Christensen H N. Albritton L M. Kakuda D K. MacLeod C L. Gene-product designations for amino acid transporters. Journal of Experimental Biology. 196:51-7, 1994.
53. Agu R. Dang H V. Jorissen M. Williems T. Vandonink S. Van Lint J. Vandenheede J V. Kinget R. Verbeke N. In vitro polarized transport of L-phenylalanine in human nasal epithelium and partial characterization of the amino acid transporters involved. Pharmaceutical Research. 20:1125-32, 2003.
54. Maulen N P. Henriquez E A. Kempe S. Carcamo J G. Schmid-Kotsas A. Bachem M. Grunert A. Bustamante M E. Nualart F. Vera J C. Up-regulation and polarized expression of the sodium-ascorbic acid transporter SVCT1 in post-confluent differentiated CaCo-2 cells. Journal of Biological Chemistry. 278(11):9035-41, 2003.
55. Michels A J. Joisher N. Hagen T M. Age-related decline of sodium-dependent ascorbic acid transport in isolated rat hepatocytes. Archives of Biochemistry & Biophysics. 410(1):112-20, 2003.
56. Sotiriou S. Gispert S. Cheng J. Wang Y. Chen A. Hoogstraten-Miller S. Miller G F. Kwon O. Levine M. Guttentag S H. Nussbaum R L. Ascorbic-acid transporter Slc23a1 is essential for vitamin C transport into the brain and for perinatal survival. Nature Medicine. 8(5):514-7, 2002.
57. MacDonald L. Thumser A E. Sharp P. Decreased expression of the vitamin C transporter SVCT1 by ascorbic acid in a human intestinal epithelial cell line. British Journal of Nutrition. 87(2):97-100, 2002.
58. Castro M. Caprile T. Astuya A. Millan C. Reinicke K. Vera J C. Vasquez O. Aguayo L G. Nualart F. High-affinity sodium-vitamin C co-transporters (SVCT) expression in embryonic mouse neurons. Journal of Neurochemistry. 78(4):815-23, 2001.
59. M. Maresca, R. Mahfoud, N. Garmy, and J. Fantini. The mycotoxin deoxynivalenol affects nutrient absorption in human intestinal epithelial cells. American Journal of Clinical Nutrition 132(9):2723-31, 2002.
60. Remington: The Science and Practice of Pharmacy (ed. Alfonso R. Gennaro), 20th edition. Lippincott Williams & Wilkins, Baltimore.
61. S. Nema, R. J. Washkuhn, and R. J. Brendel. Excipients and their use in injectable products. PDA Journal of Pharmaceutical Science and Technology 51:166-171, 1997.
62. Polli, J. E., Yu, L. X., Cook, J. A., Amidon, G. L., Borchardt, R. T., Burnside, B. A., Burton, P S., Chen, M.-L., Conner, D. P., Faustino, .J., Hawi, A. A., Hussain, A. S. Joshi, H N., Kwei, G. Lee, V.H.L., Lesko, L. J., Lipper, R. A., Loper, A. E., Nerurkar, S. G., Polli, J. W. Sanvordeker, D. R., Taneja, R., Uppoor, R. S., Vattikonda, C. S., Wilding, I., and Zhang, G. (2004): Summary Workshop Report: Biopharmaceutics Classification System—Implementation Challenges and Extension Opportunities. Journal of Pharmaceutical Sciences 93:1375-1381.
63. The FDA has recently made available to the public the Inactive Ingredients Database, which lists inactive ingredients in FDA-approved drug products. The Inactive Ingredients Database can be accessed at http://www.accessdata.fda.gov/scripts/cder/iig/index.cfm . This list is searchable by ingredient name. Each database cites the maximum amount of inactive ingredient for each route/dosage form containing the inactive ingredient.

Although the description above contains many specificities, these should not be construed as limiting the scope of the invention, but as merely as providing illustrations of some of the preferred embodiments of this invention.

1. A method of enhancing uptake of active agent, said method comprising:
co-administration of active agent that is a substrate of a sodium-dependent transporter with sodium.

2. A method of enhancing uptake of active agent, said method comprising:
a dosage form containing an active agent that is a substrate of a sodium-dependent transporter and containing sodium.

3. The method of claim 2 further comprising the step of:
formulating said dosage form for oral administration to increase uptake from the gastrointestinal tract from an animal or human.

4. The method of claim 3 further comprising the step of:
formulating said dosage form with sodium chloride.

5. The method of claim 3 further comprising the step of:
formulating said dosage form with acyclovir valychenodeoxycholate.

6. The method of claim 3 further comprising the step of:
formulating said dosage form with acyclovir valychenodeoxycholate and sodium chloride.

7. A tablet containing acyclovir valychenodeoxycholate and sodium chloride.

8. The method of claim 7 further comprising the step of:
formulating said tablet with at least 0.5 milliequivalent of sodium.
